(19) United States
(12) Patent Application Publication
(10) Pub. No.: US 2023/0024072 A1
(43) Pub. Date: Jan. 26, 2023

(54) Millimeter-Scale Chip-Based Supercontinuum Generation For Optical Coherence Tomography

(71) Applicant: The Trustees of Columbia University in the City of New York, New York, NY (US)

(72) Inventors: Xingchen Ji, New York, NY (US); Michal Lipson, New York, NY (US); Diana Mojahed, New York, NY (US); Yoshitomo Okawachi, River Edge, NJ (US); Christine P Hendon, Bronx, NY (US); Alexander L Gaeta, New York, NY (US)

(21) Appl. No.: 17/812,196

(22) Filed: Jul. 13, 2022

Related U.S. Application Data
(60) Provisional application No. 63/221,399, filed on Jul. 13, 2021.

(51) Int. Cl. G02F 1/35; G02F 1/365; A61B 5/00; G02B 6/122; H01S 3/00

(52) U.S. Cl. CPC G02F 1/3528; G02F 1/365; A61B 5/0066; G02B 6/1225; H01S 3/0092; G02B 2006/12061

(57) ABSTRACT

Methods, systems, and devices are described for generating an optical signal. An example device may comprise a chip and a waveguide disposed on the chip and comprising silicon nitride. The waveguide may be configured to generate, based on nonlinear effects applied to a pump signal from a pump laser, an optical signal having a broader spectrum than the pump signal. The waveguide may have a width and a height such that the optical signal has near zero group-velocity-dispersion.

### CROSS-REFERENCE TO RELATED APPLICATIONS

This application claims benefit to U.S. Provisional Application No. 63/221,399, filed Jul. 13, 2021, the entirety of which is herein incorporated by reference for any and all purposes.

### GOVERNMENT SUPPORT CLAUSE

This invention was made with government support under HL127776 awarded by the National Institute of Health and under FA9550-15-1-0303 awarded by the United States Air Force/Air Force Office of Scientific Research. The government has certain rights in the invention.

### BACKGROUND

Supercontinuum sources for optical coherence tomography (OCT) have raised great interest as they provide broad bandwidth to enable high resolution and high power to improve imaging sensitivity. Commercial fiber-based supercontinuum systems require high pump powers to generate broad bandwidth and customized optical filters to shape/attenuate the spectra. They also have limited sensitivity and depth performance. Thus, there is a need for more sophisticated supercontinuum devices.

### SUMMARY

Methods, systems, and devices are described for generating an optical signal. An example device may comprise a chip and a waveguide disposed on the chip and comprising silicon nitride. The waveguide may be configured to generate, based on nonlinear effects applied to a pump signal from a pump laser, an optical signal having a broader spectrum than the pump signal. The waveguide may have a width and a height such that the optical signal has near zero group-velocity-dispersion.

An example system may comprise a pump laser configured to generate a pump signal, an optical coherence tomography (OCT) scanner, and a device comprising a chip and a waveguide disposed on the chip and comprising silicon nitride. The waveguide may be configured to generate, based on nonlinear effects applied to a pump signal from a pump laser, an optical signal having a broader spectrum than the pump signal. The waveguide may have a width and a height such that the optical signal has near zero group-velocity-dispersion. The device may be configured to generate an optical signal based on the pump signal and provide the optical signal to the OCT scanner.

An example method may comprise generating, by a pump laser, a pump signal. The method may comprise providing the pump signal to a waveguide disposed on a chip. The waveguide may comprise silicon nitride. The method may comprise generating, based on nonlinear effects caused by the waveguide to the pump signal, an optical signal having a broader spectrum than the pump signal. The waveguide may have a width and a height such that the optical signal has near zero group-velocity-dispersion.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

Additional advantages will be set forth in part in the description which follows or may be learned by practice. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate embodiments and together with the description, serve to explain the principles of the methods and systems.

The file of this patent or application contains at least one drawing/photograph executed in color. Copies of this patent or patent application publication with color drawing(s)/photograph(s) will be provided by the Office upon request and payment of the necessary fee.

FIG. 1A shows mode simulation of an example 730 nm tall and 840 nm wide waveguide showing that the fundamental transverse electric (TE) mode is highly confined in the chosen geometry.

FIG. 1B shows simulated group velocity dispersion (GVD) of an example waveguide that provides close to zero-GVD near 1300 nm, which allows us to directly pump and efficiently generate broadband supercontinuum at this wavelength without any post-filtering.

FIG. 1C shows a top view of an example optical microscope image of multiple 5 cm long high confinement waveguides fabricated on the same chip. The zoom-in shows that the fabricated waveguide only occupies an area of 1×1 mm².

FIG. 2 shows measured supercontinuum spectrum generated using the Si₃N₄ waveguide. The spectrum has a 30-dB bandwidth of 445 nm covering 990 nm to 1435 nm and a flat 3-dB bandwidth spanning 1264 nm to 1369 nm with an input pump pulse energy of 25 pJ.

FIG. 3 shows a schematic of an example fiber-coupled spectral domain (SD) OCT system with a supercontinuum source generated by the Si₃N₄ waveguide.

FIG. 4 shows volumetric 3D scan of healthy breast parenchyma acquired with an example Si₃N₄ chip light source. Below, representative OCT B-scans from the 3D volume with corresponding H&E histology. Visualized parenchyma structures included ducts, cysts, lobules, adipose, and stroma. Scale bar=500 μm.

FIG. 5 shows the axial resolution of an example Si₃N₄-OCT system was calculated by measuring the full-width half-maximum (FWHM) of the axial point spread function (PSF) of the Gaussian-fitted profile. The axial resolution is 7.45 μm in air (4.97 μm in tissue).

FIG. 6 shows a sensitivity fall-off measurement of an example Si₃N₄-OCT system. The measured 6-dB fall-off is 1.81 mm.

FIG. 7 shows a comparison of the disclosed techniques with state-of-the-art commercial supercontinuum systems (SuperK Extreme). In our system, the measured sensitivity is 105 dB and 6-dB sensitivity roll-off is 1.81 mm with 300 μW power on the sample at an A-line rate of 28 kHz. For comparison, a commercial supercontinuum system (SuperK Extreme) show 95 dB sensitivity and 1.25 mm 6-dB sensitivity roll-off with 4 mW of power on the sample at an A-line rate of 40 kHz.

FIG. 8 shows supercontinuum generation simulation in an example 5-cm-long Si₃N₄ waveguide. The top portion shows simulated spectra for 128 independent initiations. The bottom portion shows calculated first-order mutual coherence g₁₂.

FIG. 9 shows supercontinuum generation simulation with an example Si₃N₄ platform using a pump wavelength at 1.06 μm can emit a coherent spectrum spanning approximately 800 nm to 1400 nm. This simulated waveguide has a cross-section of 730 nm×700 nm and 6 mm long. The top portion shows simulated group velocity dispersion (GVD). The pump is centered at 1060 nm with 100 fs pulse duration and 75 pJ pulse energy. The middle portion shows simulated 641 spectra for 128 independent initiations. The bottom portion shows calculated first-order mutual coherence.

FIG. 10 is a block diagram illustrating an example computing device.

FIG. 11 shows imaging of 10 layers of scotch tap of an Si₃N₄ Chip-OCT system at 800 nm.

FIG. 12 shows imaging of breast tissue by an Si₃N₄ Chip-OCT system at 800 nm.

### DETAILED DESCRIPTION OF ILLUSTRATIVE EMBODIMENTS

Disclosed herein is a supercontinuum platform based on, for example, a 1 mm² Si₃N₄ photonic chip for OCT. We directly pump and efficiently generate supercontinuum near 1300 nm without any post-filtering using this chip. With 25 pJ pump pulse, we generate a broadband spectrum with flat 3-dB bandwidth of 105 nm. In some scenarios, the bandwidth may be in a range of about tens of nm to about hundreds of nm, such as range of about 10 nm to about 100 nm, about 10 nm to about 150 nm, about 10 nm to about 200 nm, about 10 nm to about 300 nm, about 10 nm to about 400 nm, or about 10 nm to about 500 nm. The value of 105 nm is just an example. Integrating the chip into a spectral domain OCT system, we achieve 105-dB sensitivity and 1.81 mm 6-dB sensitivity roll-off with merely 300 μW optical power on sample. In some scenarios, the optical power may be in a range of tens of nW to tens of mW, such as a range of about 10 nW to about 20 mW, about 10 nW to about 30 mW, about 10 nW to about 40 mW, about 10 nW to about 50 mW, about 10 nW to about 60 mW, about 10 nW to about 70 mW, about 10 nW to about 80 mW, or about 10 nW to about 90 mW. The value of 300 μW is just an example. We image breast tissue to demonstrate strong imaging performance. We anticipate our chip will pave the way towards portable OCT and incorporating integrated photonics platforms into optical imaging technologies.

Disclosed herein is a millimeter-scale supercontinuum source based on a Si₃N₄ photonic chip for OCT imaging that provides improved imaging performance compared to the state-of-the-art commercial supercontinuum source.

Optical coherence tomography (OCT) is a high-resolution, label-free, three-dimensional optical imaging modality (1). OCT has become the standard-of-care in medical specialties such as ophthalmology (1-6), dermatology (7-9), and is an emerging imaging technology in other areas such as gastroenterology (10-12) and breast cancer imaging (13-15). Supercontinuum light sources for OCT offer broad bandwidth and excellent spatial coherence (16-22), but they require very high power to achieve broad bandwidth and strong performance in terms of sensitivity and sensitivity roll-off range, and the spectrum needs to be shaped and attenuated with conventional bulk optical filters. Fibers used in commercial systems require high optical pump powers (23) to generate supercontinuum which in turn requires complex optical filters to attenuate the power to meet radiation safety standards for medical imaging and not saturate or damage the camera detector. In addition, commercial supercontinuum sources can suffer from excess noise which limits OCT performance in terms of sensitivity and depth performance (sensitivity roll-off range) (24-26). The excess noise increases exponentially with imaging speed (27), which is particularly disadvantageous in clinical settings where fast imaging speeds are required. Moreover, commercial supercontinuum sources are bulky in size, not to mention that they require additional optical filtering setups, which further limits their practicality. Efforts have been made to develop supercontinuum sources for OCT centered at 1300 nm (which is one of the common imaging wavelengths (19, 23-25, 28-32)), all-normal-dispersion (ANDi) fibers have also been proposed to reduce the excess noise, but they still need filters and have limited sensitivity and depth performance (sensitivity roll-off) (25, 26, 33, 34) and have not been widely used in commercial systems. For example, the NKT Photonics SuperK Extreme, the most common commercial supercontinuum source used in OCT imaging applications, has high excess noise leading to low sensitivity and poor sensitivity roll-off performance, especially when imaging at high speeds. With 4 mW power on the sample, the maximum sensitivity achieved is 95 dB and the 6-dB sensitivity roll-off is limited to 1.25 mm (24). Furthermore, due to the low efficiency of supercontinuum generation in commercial systems, the supercontinuum source needs hundreds of milliwatts to a few watts of average pump power and several tens of nanojoules pulse energy at a 1064 nm pump wavelength. Consequently, custom filters and attenuators are required to shape the output spectrum and attenuate the output power to be safely used for imaging. Recently, Yuan et al. (35) showed that excess noise can be reduced in commercial OCT systems. However, they constrain the working condition of the laser, require careful tuning of the reference arm power, and do not fundamentally solve the problem of high excess noise when using commercial optical fibers.

### Results

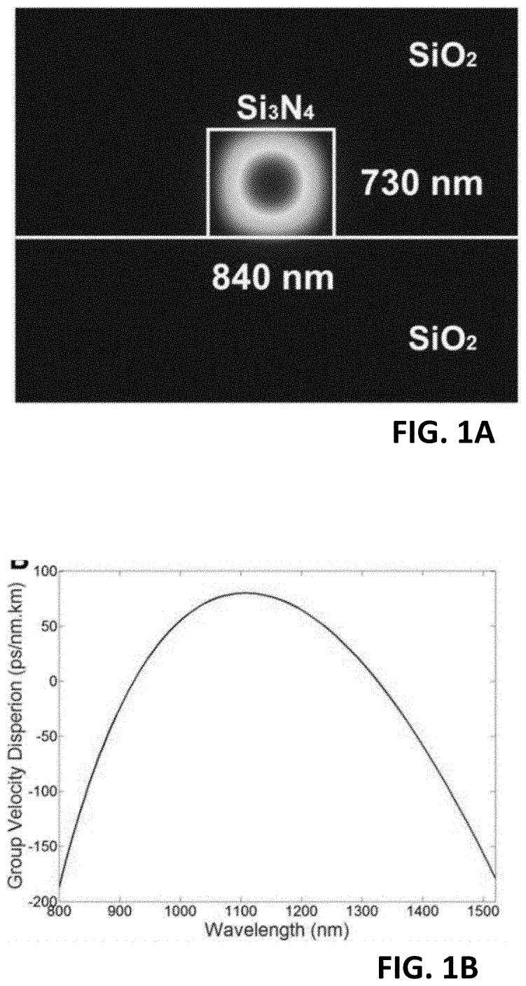

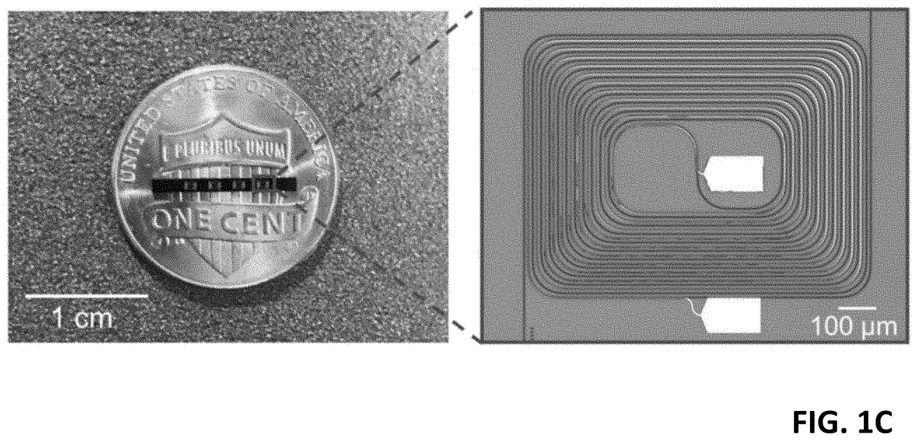

We demonstrate a supercontinuum light source for OCT imaging in a compact 1 mm×1 mm silicon nitride (Si₃N₄) photonic chip. Si₃N₄ combines the beneficial characteristics of a high refractive index, a high nonlinear parameter, wide transparency window (from visible to mid-IR), and compatibility with large-scale semiconductor manufacturing (36-38). Due to the high optical confinement and intrinsic nonlinearity in Si₃N₄, the waveguide has a nonlinearity parameter of 2710 W⁻¹km⁻¹, which is 100 times larger than that of highly nonlinear fibers commonly used in commercial supercontinuum systems (39, 40), enabling power efficient supercontinuum generation with no additional optical filtering to shape or attenuate the spectrum. In some scenarios, the waveguide may have a nonlinearity parameter in a range of about 1400 to about 3200 W⁻¹km⁻¹ (or 1400 to 3200 W⁻¹km⁻¹). The parameter value of 2710 W⁻¹km⁻¹ is just one of many examples. The Si₃N₄ platform allows the dispersion to be tailored in the waveguide simply by adjusting the waveguide cross section, allowing for flexibility in the operating wavelength. This allows for power-efficient supercontinuum generation, enabling us to obtain better sensitivity and sensitivity roll-off range with a fraction of the power on the sample. We design the waveguide cross-section to have a zero-group velocity dispersion (GVD) point near 1300 nm (mode simulation shown in FIG. 1A and simulated GVD in FIG. 1B). Using a 5-cm long waveguide with a cross-section of 730×840 nm, we achieve a broadband and spectrally flat supercontinuum spectrum with 200-fs pump pulses centered at 1300 nm with pump pulse energies of 25 pJ, which corresponds to an average pump power of 2 mW. The pump laser is commercially available, with a repetition rate of 80 MHz and the average power of about 4 mW before coupling to the chip. The supercontinuum spectrum generated in the Si₃N₄ chip is using the transverse electric (TE) mode as shown in FIG. 1A and directly measured with an optical spectrum analyzer (OSA). The result is shown in FIG. 2. The spectral broadening is mainly due to self-phase modulation (SPM), resulting in a coherent low noise spectrum. The 30-dB bandwidth spans 990 nm to 1435 nm. The 3-dB bandwidth ranges from 1264 nm to 1369 nm (105 nm), corresponding to an axial resolution of 7.28 μm in air (4.86 μm in tissue). We show the numerical modeling of supercontinuum generation and calculated coherence in Si₃N₄ waveguide in the Methods section. The fabricated waveguide only occupies an area of 1 mm² as shown in FIG. 1C. Given these output spectral characteristics for imaging, no additional optical filtering is needed to shape or attenuate the spectrum.

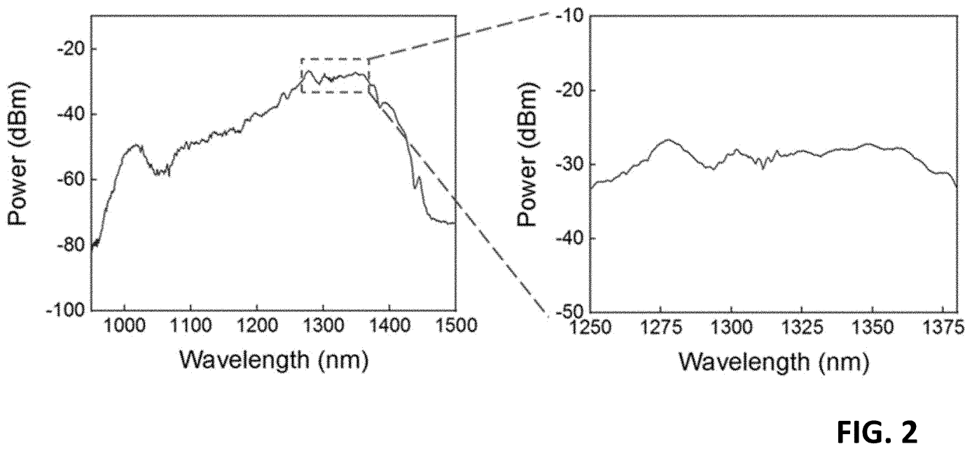

We integrate our Si₃N₄ chip into a fiber-coupled spectral domain (SD) OCT system centered at 1300 nm. The schematic diagram of our system is shown in FIG. 3. The output light from our Si₃N₄ chip is sent directly to the OCT interferometer through a circulator. The interferometer consists of a reference arm and a sample arm. In the reference arm, a glass block is used to minimize distortion caused by dispersion. In the sample arm, a two-axis galvanometer scanner is used to scan the beam, and a telecentric scan lens (NA=0.055, EFL=36 mm) focuses the beam onto the sample. The backscattered signals from the two interferometer arms are acquired by the spectrometer, which has 1024 pixels covering a spectral range of 1199.5 nm to 1367 nm and an imaging depth range of 2.52 mm.

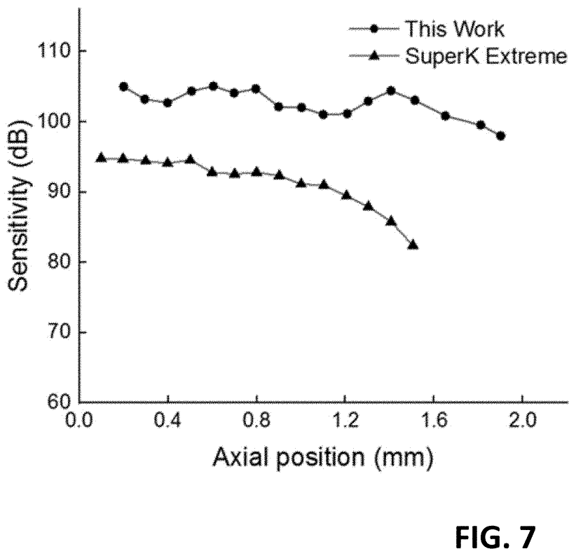

Here, we measure the performance of the Si₃N₄-OCT system and demonstrate 105 dB sensitivity and 1.81 mm 6-dB sensitivity roll-off with merely 300 μW power on the sample at an A-line rate of 28 kHz. For comparison, commercial supercontinuum systems show 95 dB and 1.25 mm 6-dB sensitivity roll-off with 4 mW of power on the sample at an A-line rate of 40 kHz (SuperK Extreme) (24) and 81 dB sensitivity and 1.20 mm 6-dB sensitivity roll-off with 4 mW power on the sample at an A-line rate of 25 kHz (SuperK Compact) (25), respectively (see Table 1 and FIG. 7). We measure the 6-dB sensitivity roll-off range to be 1.81 mm in our system, compared with the 6-dB sensitivity roll-off range of 1.25 mm using a state-of-art SuperK Extreme system that needs 10× more power on the sample. The sensitivity is measured using the method described previously (41). Our measured sensitivity is close to the theoretical shot noise limited prediction assuming a spectrometer detection efficiency of 0.4. The axial resolution is measured to be 7.45 μm in air (4.97 μm in tissue), which is in good agreement with the theoretical axial resolution of 7.41 μm (in air), accounting for the wavelength detection range of the spectrometer (See Methods).

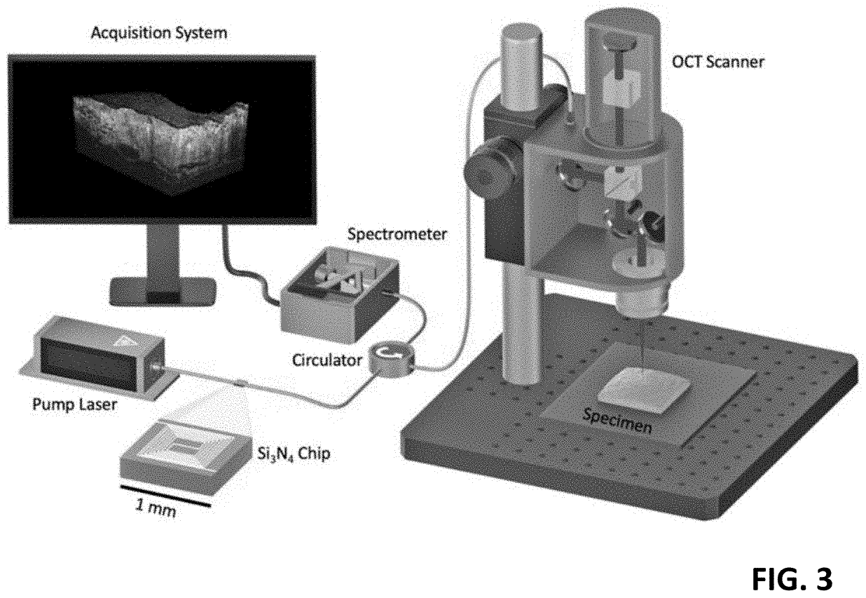

We demonstrate the ability of our Si₃N₄ chip-OCT system to resolve diverse microscopic biological tissue architecture by imaging healthy human breast tissue. The human breast tissue samples were collected from patients undergoing mastectomy procedures at Columbia University Irving Medical Center (CUIMC) and were not required for diagnosis and handled in accordance with Code of Federal Regulations 45CFR46. The specimens were fixed in formalin and imaged ex vivo within 24 hours of surgical excision. Imaging was performed at an A-line rate of 28 kHz. The total acquisition time of a single image (OCT B-scan) is 35 ms and the imaging depth is 2.52 mm. FIG. 4 shows a volumetric 3D scan of healthy breast tissue, which demonstrates important microscopic structural features of healthy breast tissue such as milk ducts, lobules, adipose (fat), and stroma (connective tissue). We did not average or pre-process these images. We process the OCT images from the raw data by performing background subtraction, linear-k interpolation, apodization, and digital dispersion compensation.

### Discussion

We demonstrate a supercontinuum light source for OCT imaging in a compact 1 mm×1 mm Si₃N₄ photonic chip that can be directly pumped at 1300 nm and does not require any optical filtering to shape or attenuate the spectrum. Our Si₃N₄ chip platform achieves 105 dB sensitivity and 1.81 mm 6-dB sensitivity roll-off with merely 300 μW optical power on the sample. The same sensitivity would require 100 times more optical power using a state-of-the-art commercial supercontinuum source. The central wavelength of 1300 nm used here is particularly suitable for imaging applications where deeper penetration depths are needed, such as breast cancer, cardiovascular, or dermatology research. Nevertheless, with flexible dispersion-engineering enabled by integrated photonics, the source's design can be easily modified to generate other spectral ranges, such as 1 μm or 800 nm. Silicon photonics for miniaturization of different building blocks of OCT systems has been explored recently by various groups. For example, Yurtsever et al. (42) demonstrated a silicon-based integrated interferometer which had a sensitivity of 62 dB with 115 μW power on the sample. Schneider et al. (43) realized an integrated interferometer and an integrated photodiode which had a sensitivity of 64 dB with 300 μW power on the sample, while Eggleston et al. (44) also demonstrated an integrated interferometer with integrated balanced photodiodes and a co-packaged MEMS mirror which had a sensitivity of 90 dB with 550 μW power on the sample. Nguyen et al. (45) showed an integrated optics spectrometer which has a sensitivity of 75 dB, and Akca et al. (46) fabricated a miniature spectrometer and a beam splitter system which had a sensitivity of 74 dB with 500 μW power on the sample. More recently, Rank et al. (6) demonstrated an arrayed waveguide grating which had a sensitivity of up to 91 dB with 830 μW power on the sample.

Here, we demonstrate a miniature supercontinuum light source which has a sensitivity of 105 dB with 300 μW power on the sample. Although we use an off-chip femtosecond pump laser in the present setup, efforts are being made towards miniaturization of mode-locked lasers (47). Together with the efforts of miniaturizing different OCT building blocks using silicon photonics and imaging probes (48-53), there is potential towards the realization of a high performance, low-cost, and fully integrated OCT system.

Supercontinuum generation using integrated waveguides with different material platforms has been extensively studied over the past decade (54-69). Si₃N₄ has the benefit of being complementary metal-oxide-semiconductor (CMOS) process-compatible, which can leverage large-scale semiconductor manufacturing at low cost. Further, it combines the beneficial characteristics of ultra-low loss (which allows fabrication of longer waveguide length and lower pump powers), a high-index contrast between the waveguide and the cladding index (which yields a large effective nonlinearity and the ability to tailor the dispersion of the waveguide), and a wide transparency window (from visible to mid-IR), which covers the wavelength windows of OCT imaging such as 800 nm, 1000 nm, 1300 nm, and 1700 nm. These characteristics make Si₃N₄ a good candidate for OCT imaging applications. Unlike highly nonlinear fibers, achieving proper group-velocity dispersion requires careful engineering of the dimensions, porosity, and spacing of the interior air holes. Dispersion engineering in integrated photonics is more easily achievable, and advanced dispersion engineering techniques, such as tapering the waveguide width to shift the dispersive wave phase-matching wavelength (70), can be further applied in integrated photonics to achieve flat spectra. Our experiment demonstrates that the integrated Si₃N₄ photonics platform is promising for OCT imaging, and we anticipate seeing other integrated photonics platforms being utilized for biomedical imaging applications.

### Materials and Methods

Si₃N₄ Chip Fabrication

We demonstrate a low-loss 5-cm long Si₃N₄ waveguide in 1 mm² area fabricated on a 4-inch silicon wafer designed for OCT imaging. Starting from the silicon wafer, we thermally grow a 4-μm thick oxide layer as the bottom cladding. Si₃N₄ is deposited using low-pressure chemical vapor deposition (LPCVD) in two steps and annealed at 1200° C. in an argon atmosphere for 3 hours in between steps. After Si₃N₄ deposition, we deposit a SiO₂ hard mask using plasma-enhanced chemical vapor deposition (PECVD). We pattern our devices using electron beam lithography. Ma-N 2403 resist was used to write the pattern and the nitride film was etched in an inductively coupled plasma reactive ion etcher (ICP RIE) using a combination of CHF₃, N₂, and O₂ gases. After stripping the oxide mask, we anneal the devices again to remove residual N—H bonds in the Si₃N₄ film. We clad the devices with 500 nm of high-temperature silicon dioxide (HTO) deposited at 800° C. followed by 2 μm of SiO₂ using PECVD. A deep etched facet and an inverse taper is designed and used to minimize the edge coupling loss.

Nonlinearity Calculation

The nonlinear coefficient γ is often used to determine the degree of nonlinearity and is given by Eqn. 1 (71):

$$γ = \frac{2 π {n}_{2}}{λ {A}_{e f f}} \qquad ( 1 )$$

where n₂ is the nonlinear Kerr coefficient of the material, λ is the wavelength, and A_{eff} is the effective mode area. The n₂ of Si₃N₄ is 2.45×10¹⁵ cm²/W, which is 10 times larger than that of silica (72). The A_{eff} of our waveguide is calculated to be 0.43 μm². The small A_{eff} and large n₂ of Si₃N₄ leads to the nonlinearity parameter γ value of 2710 W⁻¹km⁻¹, which is more than 100 times that of high nonlinear fibers and more than 1000 times that of standard single mode fibers (40).

Resolution, Sensitivity, and Sensitivity Roll-Off

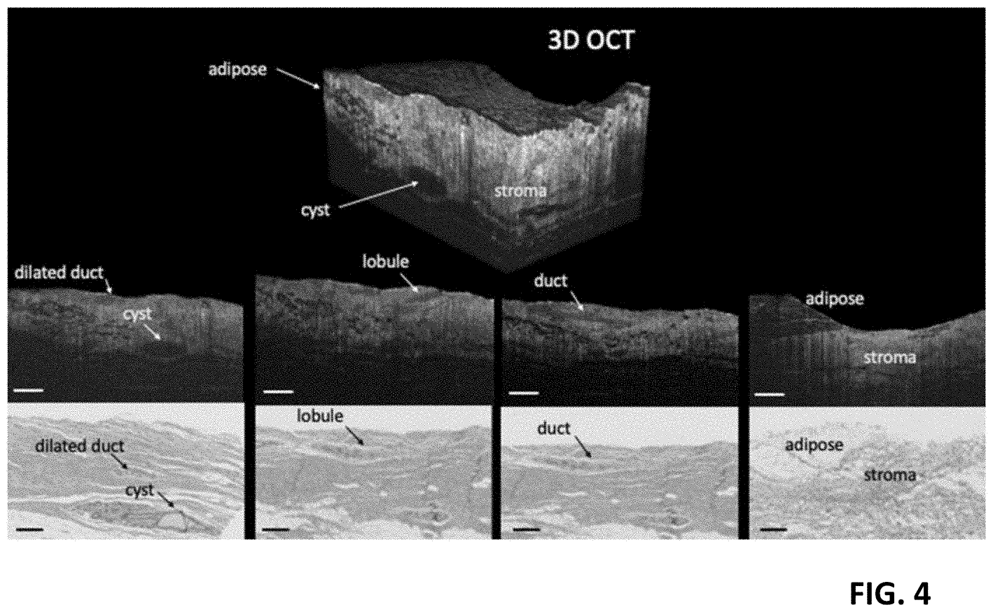

The axial resolution of our chip-based supercontinuum source is 7.45 μm in air (4.97 μm in tissue). We measured the axial resolution by calculating the full-width half-maximum (FWHM) of the axial point spread function (PSF) of a flat mirror at the focal plane of the sample arm (e.g., FIG. 5). Our measurement is in good agreement with the theoretical axial resolution of 7.41 μm (in air), accounting for the wavelength detection range of the spectrometer using Eqn. 2:

$$Axial resolution = \frac{2 ln 2}{π} \cdot \frac{λ \frac{2}{0}}{Δ {λ}_{F W H M}} \qquad ( 2 )$$

where λ₀ is the central wavelength and Δλ_{FWHM} is the FWHM of the spectrum.

The signal-to-noise ratio (SNR), used interchangeably with sensitivity in the literature, is directly proportional to the power reflected by the sample divided by the noise of the system (Eqn. 3) (76). The noise of the system (σ2/sys) is defined as σ_{sys}²=σ_{excess}²+σₛₕₒₜ²+σ_{rec}², where σ_{excess}² represents the excess noise, σₛₕₒₜ² represents the shot noise, and σ_{rec}² represents the receiver noise. The receiver noise term, which represents the sum of the dark noise of the detector and read-out noise of the circuit, is negligible in comparison to the other terms and thus omitted from Eqn. 3.

$$SNR = \frac{< {S}_{O C T}^{2} >}{{σ}_{s y s}^{2}} = \frac{\frac{1}{16} {( \frac{η {P}_{0} τ}{h {v}_{0} N} )}^{2} {R}_{R} {R}_{S}}{{σ}_{e x c e s s}^{2} + {σ}_{s h o t}^{2}} \qquad ( 3 )$$

Combining the supercontinuum generated by our Si₃N₄ chip with a fiber-based SD-OCT system, we achieve 105 dB sensitivity. The measured sensitivity is in agreement with the theoretical shot noise limited prediction assuming a spectrometer detection efficiency is 0.4.

In SD-OCT, specifically, the sensitivity decreases along the imaging depth.

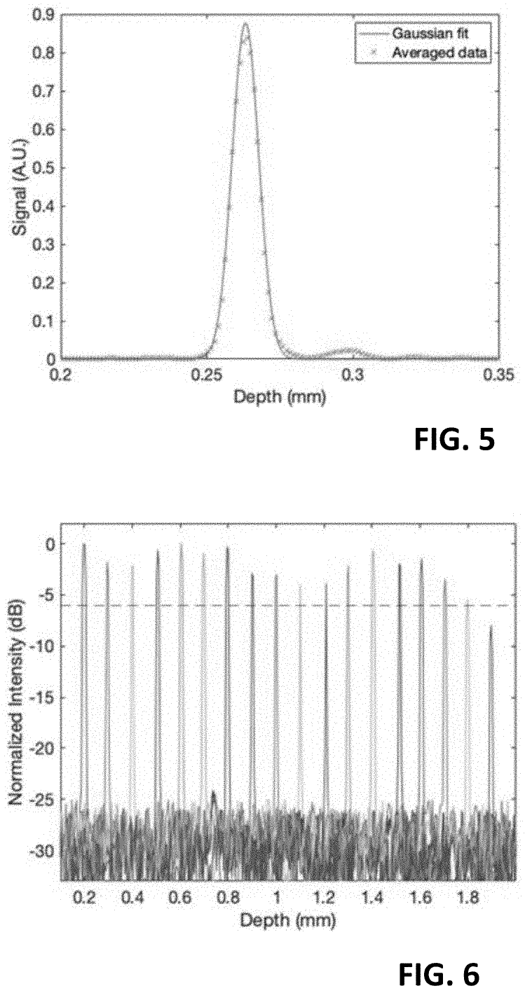

There is a reduction in fringe visibility that is more predominant at higher fringe frequencies due to the finite resolution of the spectrometer. The standard method of characterizing the sensitivity fall-off is by measuring the depth at which the signal decreases by 6-dB. Here we measure the sensitivity fall-off by placing a flat mirror at the focal plane of the sample arm while moving the reference arm mirror at fixed increments and measuring the PSF at each position until a 6-dB sensitivity fall-off is observed. We measured 1.81 mm 6-dB sensitivity roll-off with 300 μW power on the sample at an A-line rate of 28 kHz (e.g., FIG. 6). We observed noticeably stronger sensitivity fall-off performance for our chip-based supercontinuum source when compared to commercial supercontinuum sources (e.g., FIG. 7).

Numerical Modeling of Supercontinuum Generation in Si₃N₄ Waveguide

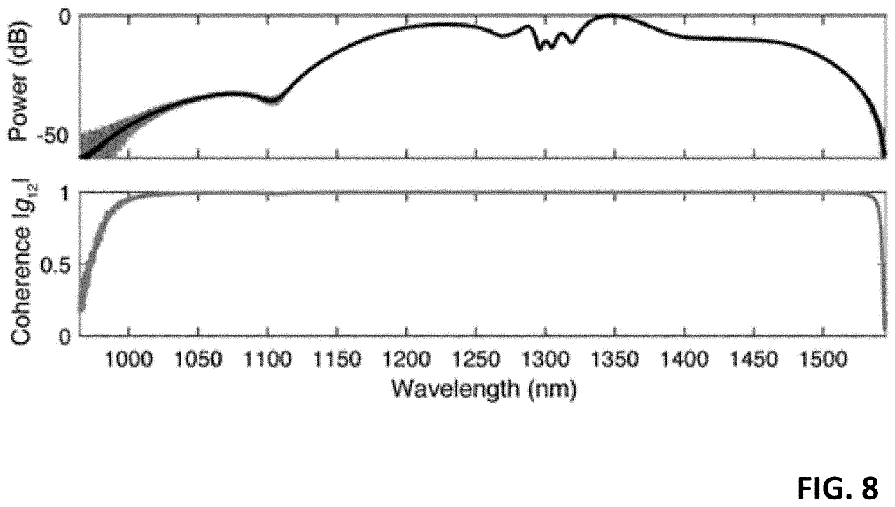

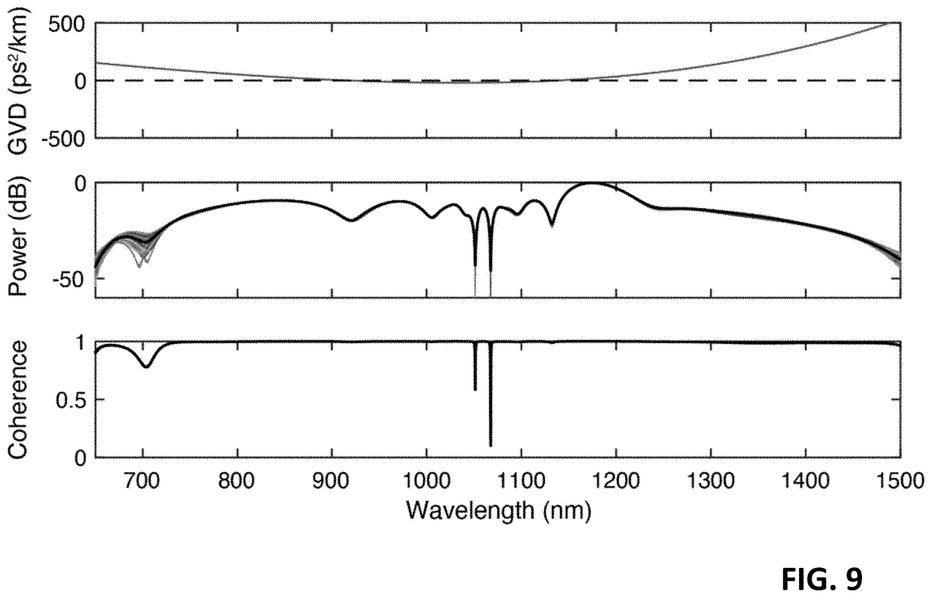

We have performed numerical modeling of the pulse propagation dynamics in a silicon nitride (Si₃N₄) waveguide using parameters similar to our experiment. The dispersion of the waveguide is simulated using a finite element mode solver. The waveguide cross section is 730×840 nm. We model pulse propagation in a 5-cm long waveguide by solving the nonlinear Schrodinger equation using the split-step Fourier method (73), taking into account higher-order dispersion, third-order nonlinearity, and self-steepening. The input pulse has a 200-fs duration centered at 1300 nm with a pulse energy of 25 pJ. The simulated spectrum spans 500 nm. Discrepancies in the spectral profile between the experiment and simulation are attributed to the difference in the simulated dispersion from the actual waveguide dispersion due to fabrication tolerances. In addition, we analyze the spectral coherence of the generated output by performing 128 independent simulations using pulses seeded with quantum shot noise and calculating the first-order mutual coherence g₁₂ (73-75). FIG. 8 shows the simulated spectrum and the calculated coherence. Our results indicate that the spectrum exhibits near-unity spectral coherence over the entire spectral bandwidth. We have also showed it is possible to pump at 1.06 μm and generated a broadband highly spectrally coherent supercontinuum covering the OCT imaging window of 1300 nm with dispersion engineering in FIG. 9.

### REFERENCES

1. D. Huang, E. A. Swanson, C. P. Lin, J. S. Schuman, W. G. Stinson, W. Chang, M. R. Hee, T. Flotte, K. Gregory, C. A. Puliafito, A. Et, Optical coherence tomography. Science. 254, 1178-1181 (1991).
2. M. R. Hee, J. A. Izatt, E. A. Swanson, D. Huang, J. S. Schuman, C. P. Lin, C. A. Puliafito, J. G. Fujimoto, Optical coherence tomography of the human retina. Arch. Ophthalmol. 113, 325-332 (1995).
3. W. Drexler, U. Morgner, R. K. Ghanta, F. X. Kartner, J. S. Schuman, J. G. Fujimoto, Ultrahigh-resolution ophthalmic optical coherence tomography. Nat. Med. 7, 502-507 (2001).
4. J. Yi, Q. Wei, W. Liu, V. Backman, H. F. Zhang, Visible-light optical coherence tomography for retinal oximetry. Opt. Lett. 38, 1796-1798 (2013).
5. R. Chopra, S. K. Wagner, P. A. Keane, Optical coherence tomography in the 2020s—outside the eye clinic. Eye. 35, 236-243 (2021).
6. E. A. Rank, R. Sentosa, D. J. Harper, M. Salas, A. Gaugutz, D. Seyringer, S. Nevlacsil, A. Maese-Novo, M. Eggeling, P. Muellner, R. Hainberger, M. Sagmeister, J. Kraft, R. A. Leitgeb, W. Drexler, Toward optical coherence tomography on a chip: in vivo three-dimensional human retinal imaging using photonic integrated circuit-based arrayed waveguide gratings. Light Sci. Appl. 10, 6 (2021).
7. T. Cao, H. L. Tey, High-definition optical coherence tomography—an aid to clinical practice and research in dermatology. J. Dtsch. Dermatol. Ges. J. Ger. Soc. Dermatol. JDDG. 13, 886-890 (2015).
8. F. E. Robles, K. C. Zhou, M. C. Fischer, W. S. Warren, Stimulated Raman scattering spectroscopic optical coherence tomography. Optica. 4, 243 (2017).
9. J. Welzel, Optical coherence tomography in dermatology: a review. Skin Res. Technol. 7, 1-9 (2001).
10. M. J. Suter, B. J. Vakoc, P. S. Yachimski, M. Shishkov, G. Y. Lauwers, M. Mino-Kenudson, B. E. Bouma, N. S. Nishioka, G. J. Tearney, Comprehensive microscopy of the esophagus in human patients with optical frequency domain imaging. Gastrointest. Endosc. 68, 745-753 (2008).
11. M. J. Gora, J. S. Sauk, R. W. Carruth, K. A. Gallagher, M. J. Suter, N. S. Nishioka, L. E. Kava, M. Rosenberg, B. E. Bouma, G. J. Tearney, Tethered capsule endomicroscopy enables less invasive imaging of gastrointestinal tract microstructure. Nat. Med. 19, 238-240 (2013).
12. K. Li, W. Liang, J. Mavadia-Shukla, H.-C. Park, D. Li, W. Yuan, S. Wan, X. Li, Super-achromatic optical coherence tomography capsule for ultrahigh-resolution imaging of esophagus. J. Biophotonics. 12, e201800205 (2019).
13. F. T. Nguyen, A. M. Zysk, E. J. Chaney, J. G. Kotynek, U. J. Oliphant, F. J. Bellafiore, K. M. Rowland, P. A. Johnson, S. A. Boppart, Intraoperative Evaluation of Breast Tumor Margins with Optical Coherence Tomography. Cancer Res. 69, 8790-8796 (2009).
14. K. M. Kennedy, R. Zilkens, W. M. Allen, K. Y. Foo, Q. Fang, L. Chin, R. W. Sanderson, J. Anstie, P. Wijesinghe, A. Curatolo, H. E. I. Tan, N. Morin, B. Kunjuraman, C. Yeomans, S. L. Chin, H. DeJong, K. Giles, B. F. Dessauvagie, B. Latham, C. M. Saunders, B. F. Kennedy, Diagnostic Accuracy of Quantitative Micro-Elastography for Margin Assessment in Breast-Conserving Surgery. Cancer Res. 80, 1773-1783 (2020).
15. D. Mojahed, R. S. Ha, P. Chang, Y. Gan, X. Yao, B. Angelini, H. Hibshoosh, B. Taback, C. P. Hendon, Fully Automated Postlumpectomy Breast Margin Assessment Utilizing Convolutional Neural Network Based Optical Coherence Tomography Image Classification Method. Acad. Radiol. 27, e81-e86 (2020).
16. L. Froehly, J. Meteau, Supercontinuum sources in optical coherence tomography: A state of the art and the application to scan-free time domain correlation techniques and depth dependent dispersion compensation. Opt. Fiber Technol. 18, 411-419 (2012).
17. K. Bizheva, B. Tan, B. MacLelan, O. Kralj, M. Hajialamdari, D. Hileeto, L. Sorbara, Sub-micrometer axial resolution OCT for in-vivo imaging of the cellular structure of healthy and keratoconic human corneas. Biomed. Opt. Express. 8, 800-812 (2017).
18. W. Drexler, Ultrahigh-resolution optical coherence tomography. J. Biomed. Opt. 9, 47-74 (2004).
19. I. Hartl, X. D. Li, C. Chudoba, R. K. Ghanta, T. H. Ko, J. G. Fujimoto, J. K. Ranka, R. S. Windeler, Ultrahigh-resolution optical coherence tomography using continuum generation in an air-silica microstructure optical fiber. Opt. Lett. 26, 608-610 (2001).
20. N. Nishizawa, H. Kawagoe, M. Yamanaka, M. Matsushima, K. Mori, T. Kawabe, Wavelength Dependence of Ultrahigh-Resolution Optical Coherence Tomography Using Supercontinuum for Biomedical Imaging. IEEE J. Sel. Top. Quantum Electron. 25, 1-15 (2019).
21. T.-A. Wang, M.-C. Chan, H.-C. Lee, C.-Y. Lee, M.-T. Tsai, Ultrahigh-resolution optical coherence tomography/angiography with an economic and compact supercontinuum laser. Biomed. Opt. Express. 10, 5687-5702 (2019).
22. X. Zeng, X. Zhang, C. Li, X. Wang, J. Jerwick, T. Xu, Y. Ning, Y. Wang, L. Zhang, Z. Zhang, Y. Ma, C. Zhou, Ultrahigh-resolution optical coherence microscopy accurately classifies precancerous and cancerous human cervix free of labeling. Theranostics. 8, 3099-3110 (2018).
23. Y.-J. You, C. Wang, Y.-L. Lin, A. Zaytsev, P. Xue, C.-L. Pan, Ultrahigh-resolution optical coherence tomography at 1.3 μm central wavelength by using a supercontinuum source pumped by noise-like pulses. Laser Phys. Lett. 13, 025101 (2015).
24. M. Maria, M. Bondu, R. D. Engelsholm, T. Feuchter, P. M. Moselund, L. Leick, O. Bang, A. Podoleanu, (San Francisco, Calif., United States, 2017), Proceedings of SPIE (Vol. 10056).
25. M. Maria, I. B. Gonzalo, T. Feuchter, M. Denninger, P. M. Moselund, L. Leick, O. Bang, A. Podoleanu, Q-switch-pumped supercontinuum for ultra-high resolution optical coherence tomography. Opt. Lett. 42, 4744-4747 (2017).
26. M. Jensen, I. B. Gonzalo, R. D. Engelsholm, M. Maria, N. M. Israelsen, A. Podoleanu, O. Bang, Noise of supercontinuum sources in spectral domain optical coherence tomography. JOSA B. 36, A154-A160 (2019).
27. X. Yao, Y. Gan, C. C. Marboe, C. P. Hendon, Myocardial imaging using ultrahigh-resolution spectral domain optical coherence tomography. J. Biomed. Opt. 21, 061006 (2016).
28. P.-L. Hsiung, Y. Chen, T. H. Ko, J. G. Fujimoto, C. J. S. de Matos, S. V. Popov, J. R. Taylor, V. P. Gapontsev, Optical coherence tomography using a continuous-wave, high-power, Raman continuum light source. Opt. Express. 12, 5287-5295 (2004).
29. K. Bizheva, R. Pflug, B. Hermann, B. Považay, H. Sattmann, P. Qiu, E. Anger, H. Reitsamer, S. Popov, J. R. Taylor, A. Unterhuber, P. Ahnelt, W. Drexler, Optophysiology: Depth-resolved probing of retinal physiology with functional ultrahigh-resolution optical coherence tomography. Proc. Natl. Acad. Sci. 103, 5066-5071 (2006).
30. S. Ishida, N. Nishizawa, Quantitative comparison of contrast and imaging depth of ultrahigh-resolution optical coherence tomography images in 800-1700 nm wavelength region. Biomed. Opt. Express. 3, 282-294 (2012).
31. A. Aguirre, N. Nishizawa, J. Fujimoto, W. Seitz, M. Lederer, D. Kopf, Continuum generation in a novel photonic crystal fiber for ultrahigh resolution optical coherence tomography at 800 nm and 1300 nm. Opt. Express. 14, 1145-1160 (2006).
32. F. Spöler, S. Kray, P. Grychtol, B. Hermes, J. Bornemann, M. Först, H. Kurz, Simultaneous dual-band ultra-high resolution optical coherence tomography. Opt. Express. 15, 10832-10841 (2007).
33. A. M. Heidt, J. S. Feehan, J. H. V. Price, T. Feurer, Limits of coherent supercontinuum generation in normal dispersion fibers. JOSA B. 34, 764-775 (2017).
34. A. M. Heidt, A. Hartung, G. W. Bosman, P. Krok, E. G. Rohwer, H. Schwoerer, H. Bartell, Coherent octave spanning near-infrared and visible supercontinuum generation in all-normal dispersion photonic crystal fibers. Opt. Express. 19, 3775-3787 (2011).
35. W. Yuan, J. Mavadia-Shukla, J. Xi, W. Liang, X. Yu, S. Yu, X. Li, Optimal operational conditions for supercontinuum-based ultrahigh-resolution endoscopic OCT imaging. Opt. Lett. 41, 250-253 (2016).
36. D. J. Moss, R. Morandotti, A. L. Gaeta, M. Lipson, New CMOS-compatible platforms based on silicon nitride and Hydex for nonlinear optics. Nat. Photonics. 7, 597-607 (2013).
37. I. Goykhman, B. Desiatov, U. Levy, Ultrathin silicon nitride microring resonator for biophotonic applications at 970 nm wavelength. Appl. Phys. Lett. 97, 081108 (2010).
38. Q. Wilmart, H. El Dirani, N. Tyler, D. Fowler, S. Malhouitre, S. Garcia, M. Casale, S. Kerdiles, K. Hassan, C. Monat, X. Letartre, A. Kamel, M. Pu, K. Yvind, L. Oxenlowe, W. Rabaud, C. Sciancalepore, B. Szelag, S. Olivier, A Versatile Silicon-Silicon Nitride Photonics Platform for Enhanced Functionalities and Applications. Appl. Sci. 9, 255 (2019).
39. Y. Okawachi, M. Yu, J. Cardenas, X. Ji, M. Lipson, A. L. Gaeta, Coherent, directional supercontinuum generation. Opt. Lett. 42, 4466 (2017).
40. Y. Yamamoto, Y. Tamura, T. Hasegawa, Silica-Based Highly Nonlinear Fibers and Their Applications. SEI Tech Rev. 83, 15-20 (2016).
41. P. Cimalla, J. Walther, M. Mehner, M. Cuevas, E. Koch, Simultaneous dual-band optical coherence tomography in the spectral domain for high resolution in vivo imaging. Opt. Express. 17, 19486-19500 (2009).
42. G. Yurtsever, N. Weiss, J. Kalkman, T. G. van Leeuwen, R. Baets, Ultra-compact silicon photonic integrated interferometer for swept-source optical coherence tomography. Opt. Lett. 39, 5228-5231 (2014).
43. S. Schneider, M. Lauermann, P.-I. Dietrich, C. Weimann, W. Freude, C. Koos, Optical coherence tomography system mass-producible on a silicon photonic chip. Opt. Express. 24, 1573-1586 (2016).
44. M. S. Eggleston, F. Pardo, C. Bolle, B. Farah, N. Fontaine, H. Safar, M. Cappuzzo, C. Pollock, D. J. Bishop, M. P. Earnshaw, in Conference on Lasers and Electro-Optics (2018), paper JTh5C.8 (Optical Society of America, 2018)
45. V. D. Nguyen, B. I. Akca, K. Worhoff, R. M. de Ridder, M. Pollnau, T. G. van Leeuwen, J. Kalkman, Spectral domain optical coherence tomography imaging with an integrated optics spectrometer. Opt. Lett. 36, 1293-1295 (2011).
46. B. I. Akca, B. Považay, A. Alex, K. Worhoff, R. M. de Ridder, W. Drexler, M. Pollnau, Miniature spectrometer and beam splitter for an optical coherence tomography on a silicon chip. Opt. Express. 21, 16648-16656 (2013).
47. M. Kemiche, J. Lhuillier, S. Callard, C. Monat, Design optimization of a compact photonic crystal microcavity based on slow light and dispersion engineering for the miniaturization of integrated mode-locked lasers. AIP Adv. 8, 015211 (2018).
48. X. Li, C. Chudoba, T. Ko, C. Pitris, J. G. Fujimoto, Imaging needle for optical coherence tomography. Opt. Lett. 25, 1520-1522 (2000).
49. Y. Wu, J. Xi, L. Huo, J. Padvorac, E. J. Shin, S. A. Giday, A. M. Lennon, M. I. F. Canto, J. H. Hwang, X. Li, Robust High-Resolution Fine OCT Needle for Side-Viewing Interstitial Tissue Imaging. IEEE J. Sel. Top. Quantum Electron. 16, 863-869 (2010).
50. M. J. Gora, M. J. Suter, G. J. Tearney, X. Li, Endoscopic optical coherence tomography: technologies and clinical applications [Invited]. Biomed. Opt. Express. 8, 2405-2444 (2017).
51. W. Yuan, R. Brown, W. Mitzner, L. Yarmus, X. Li, Super-achromatic monolithic microprobe for ultrahigh-resolution endoscopic optical coherence tomography at 800 nm. Nat. Commun. 8, 1531 (2017).
52. D. Cui, K. K. Chu, B. Yin, T. N. Ford, C. Hyun, H. M. Leung, J. A. Gardecki, G. M. Solomon, S. E. Birket, L. Liu, S. M. Rowe, G. J. Tearney, Flexible, high-resolution micro-optical coherence tomography endobronchial probe toward in vivo imaging of cilia. Opt. Lett. 42, 867-870 (2017).
53. S. Kim, M. Crose, W. J. Eldridge, B. Cox, W. J. Brown, A. Wax, Design and implementation of a low-cost, portable OCT system. Biomed. Opt. Express. 9, 1232-1243 (2018).
54. N. Singh, M. Xin, D. Vermeulen, K. Shtyrkova, N. Li, P. T. Callahan, E. S. Magden, A. Ruocco, N. Fahrenkopf, C. Baiocco, B. P.-P. Kuo, S. Radic, E. Ippen, F. X. Kärtner, M. R. Watts, Octave-spanning coherent supercontinuum generation in silicon on insulator from 1.06 μm to beyond 2.4 μm. Light Sci. Appl. 7, 17131-17131 (2018).
55. J. Chiles, N. Nader, D. D. Hickstein, S. P. Yu, T. C. Briles, D. Carlson, H. Jung, J. M. Shainline, S. Diddams, S. B. Papp, S. W. Nam, R. P. Mirin, Deuterated silicon nitride photonic devices for broadband optical frequency comb generation. Opt. Lett. 43, 1527-1530 (2018).
56. D. Duchesne, M. Peccianti, M. R. E. Lamont, M. Ferrera, L. Razzari, F. Légaré, R. Morandotti, S. Chu, B. E. Little, D. J. Moss, Supercontinuum generation in a high index doped silica glass spiral waveguide. Opt. Express. 18, 923-930 (2010).
57. B. Kuyken, X. Liu, R. M. Osgood, R. Baets, G. Roelkens, W. M. J. Green, Mid-infrared to telecom-band supercontinuum generation in highly nonlinear silicon-on-insulator wire waveguides. Opt. Express. 19, 20172-20181 (2011).
58. R. Halir, Y. Okawachi, J. S. Levy, M. A. Foster, M. Lipson, A. L. Gaeta, Ultrabroadband supercontinuum generation in a CMOS-compatible platform. Opt. Lett. 37, 1685-1687 (2012).
59. F. Leo, S.-P. Gorza, S. Coen, B. Kuyken, G. Roelkens, Coherent supercontinuum generation in a silicon photonic wire in the telecommunication wavelength range. Opt. Lett. 40, 123-126 (2015).
60. J. P. Epping, T. Hellwig, M. Hoekman, R. Mateman, A. Leinse, R. G. Heideman, A. van Rees, P. J. M. van der Slot, C. J. Lee, C. Fallnich, K.-J. Boller, On-chip visible-to-infrared supercontinuum generation with more than 495 THz spectral bandwidth. Opt. Express. 23, 19596-19604 (2015).
61. B. Kuyken, T. Ideguchi, S. Holzner, M. Yan, T. W. Hänsch, J. Van Campenhout, P. Verheyen, S. Coen, F. Leo, R. Baets, G. Roelkens, N. Picqué, An octave-spanning mid-infrared frequency comb generated in a silicon nanophotonic wire waveguide. Nat. Commun. 6, 6310 (2015).
62. N. Singh, D. D. Hudson, Y. Yu, C. Grillet, S. D. Jackson, A. Casas-Bedoya, A. Read, P. Atanackovic, S. G. Duvall, S. Palomba, B. Luther-Davies, S. Madden, D. J. Moss, B. J. Eggleton, Midinfrared supercontinuum generation from 2 to 6 μm in a silicon nanowire. Optica. 2, 797-802 (2015).
63. A. Klenner, A. S. Mayer, A. R. Johnson, K. Luke, M. R. E. Lamont, Y. Okawachi, M. Lipson, A. L. Gaeta, U. Keller, Gigahertz frequency comb offset stabilization based on supercontinuum generation in silicon nitride waveguides. Opt. Express. 24, 11043-11053 (2016).
64. X. Liu, M. Pu, B. Zhou, C. J. Krückel, A. Fülöp, V. Tones-Company, M. Bache, Octave-spanning supercontinuum generation in a silicon-rich nitride waveguide. Opt. Lett. 41, 2719-2722 (2016).
65. M. A. G. Porcel, F. Schepers, J. P. Epping, T. Hellwig, M. Hoekman, R. G. Heideman, P. J. M. van der Slot, C. J. Lee, R. Schmidt, R. Bratschitsch, C. Fallnich, K.-J. Boller, Two-octave spanning supercontinuum generation in stoichiometric silicon nitride waveguides pumped at telecom wavelengths. Opt. Express. 25, 1542-1554 (2017).
66. M. Sinobad, C. Monat, B. Luther-davies, P. Ma, S. Madden, D. J. Moss, A. Mitchell, D. Allioux, R. Orobtchouk, S. Boutami, J.-M. Hartmann, J.-M. Fedeli, C. Grillet, Mid-infrared octave spanning supercontinuum generation to 8.5 μm in silicon-germanium waveguides. Optica. 5, 360-366 (2018).
67. M. Yu, B. Desiatov, Y. Okawachi, A. L. Gaeta, M. Lončar, Coherent two-octave-spanning supercontinuum generation in lithium-niobate waveguides. Opt. Lett. 44, 1222-1225 (2019).
68. B. Kuyken, M. Billet, F. Leo, K. Yvind, M. Pu, Octave-spanning coherent supercontinuum generation in an AlGaAs-on-insulator waveguide. Opt. Lett. 45, 603-606 (2020).
69. J. Lu, X. Liu, A. W. Bruch, L. Zhang, J. Wang, J. Yan, H. X. Tang, Ultraviolet to mid-infrared supercontinuum generation in single-crystalline aluminum nitride waveguides. Opt. Lett. 45, 4499-4502 (2020).
70. N. Singh, D. Vermulen, A. Ruocco, N. Li, N. Li, E. Ippen, F. X. Kärtner, F. X. Kärtner, M. R. Watts, Supercontinuum generation in varying dispersion and birefringent silicon waveguide. Opt. Express. 27, 31698-31712 (2019).
71. G. Agrawal, “Optics and Photonics” in Nonlinear Fiber Optics (Fifth Edition), (Academic Press, Boston, 2013), pp. 457-496.
72. K. Ikeda, R. E. Saperstein, N. Alic, Y. Fainman, Thermal and Kerr nonlinear properties of plasma-deposited silicon nitride/silicon dioxide waveguides. Opt. Express. 16, 12987-12994 (2008).
73. A. R. Johnson, A. S. Mayer, A. Klenner, K. Luke, E. S. Lamb, M. R. E. Lamont, C. Joshi, Y. Okawachi, F. W. Wise, M. Lipson, U. Keller, A. L. Gaeta, Octave-spanning coherent supercontinuum generation in a silicon nitride waveguide. Opt. Lett. 40, 5117-5120 (2015).
74. X. Gu, M. Kimmel, A. P. Shreenath, R. Trebino, J. M. Dudley, S. Coen, R. S. Windeler, Experimental studies of the coherence of microstructure-fiber supercontinuum. Opt. Express. 11, 2697-2703 (2003).
75. A. Ruehl, M. J. Martin, K. C. Cossel, L. Chen, H. McKay, B. Thomas, C. Benko, L. Dong, J. M. Dudley, M. E. Fermann, I. Hard, J. Ye, Ultrabroadband coherent supercontinuum frequency comb. Phys. Rev. A. 84, 011806 (2011).
76. R. Leitgeb, C. Hitzenberger, A. Fercher, Performance of fourier domain vs time domain optical coherence tomography. Opt. Express. 11, 889 (2003).

### Figures and Tables

FIGS. 1A-C show simulations and microscope image of fabricated devices.

FIG. 1A shows example mode simulation of 730 nm tall and 840 nm wide waveguide showing that the fundamental transverse electric (TE) mode is highly confined in the geometry we have chosen. FIG. 1B shows simulated group velocity dispersion (GVD) of an example waveguide that provides close to zero-GVD near 1300 nm, which allows us to directly pump and efficiently generate broadband supercontinuum at this wavelength without any post-filtering. FIG. 1C shows a top view of a optical microscope image of multiple example 5 cm long high confinement waveguides fabricated on the same chip. The zoom-in shows that the fabricated waveguide only occupies an area of 1×1 mm².

FIG. 2 shows measured supercontinuum spectrum generated using the example Si₃N₄ waveguide. The spectrum has a 30-dB bandwidth of 445 nm covering 990 nm to 1435 nm and a flat 3-dB bandwidth spanning 1264 nm to 1369 nm with an input pump pulse energy of 25 pJ.

FIG. 3 shows a schematic of an example fiber-coupled spectral domain (SD) OCT system with a supercontinuum source generated by the Si₃N₄ waveguide.

FIG. 4 shows a volumetric 3D scan of healthy breast parenchyma acquired with an example Si₃N₄ chip light source. Below, representative OCT B-scans from the 3D volume with corresponding H&E histology. Visualized parenchyma structures included ducts, cysts, lobules, adipose, and stroma. Scale bar=500 μm.

TABLE 1
Comparison of our work with state-of-the-art commercial
supercontinuum systems.

| | | | | | Filter and |
|---|---|---|---|---|---|
| | Pump | Power | | 6 dB | Spectral |
| | Wave- | on | Sensi- | Roll-off | Shaping |
| | length | Sample | tivity | Range | Needed? |
| Our Work | 1300 nm | 300 μW | 105 dB | 1.81 mm | No |
| @ 28 kHz A- | | | | | |
| line rate | | | | | |
| SuperK | 1064 nm | 4 mW | 95 dB | 1.25 mm | Yes |
| Extreme @ | | | | | |
| 40 kHz A- | | | | | |
| line rate (24) | | | | | |
| SuperK | 1064 nm | 4 mW | 81 dB | 1.1-1.2 mm | Yes |
| Compact @ | | | | | |
| 25 kHz A- | | | | | |
| line rate (25) | | | | | |

### Supplementary Materials

Resolution and Sensitivity Roll-Off

We measured an axial resolution of 7.45 μm in air (4.97 μm in tissue) by calculating the full-width half-maximum (FWHM) of the axial point spread function (PSF) of a flat mirror at the focal plane of the sample arm (e.g., FIG. 5). The standard method of characterizing the sensitivity fall-off is by measuring the depth at which the signal decreases by 6-dB. Here we measure the sensitivity fall-off by placing a flat mirror at the focal plane of the sample arm while moving the reference arm mirror at fixed increments and measuring the PSF at each position until a 6-dB sensitivity fall-off is observed. The measured 6-dB fall-off is 1.81 mm.

FIG. 5 shows the axial resolution of an example Si₃N₄-OCT system that was calculated by measuring the full-width half-maximum (FWHM) of the axial point spread function (PSF) of the Gaussian-fitted profile. The axial resolution is 7.45 μm in air (4.97 μm in tissue).

FIG. 6 shows sensitivity fall-off measurement of an example Si₃N₄-OCT system. The measured 6-dB fall-off is 1.81 mm.

FIG. 7 shows a comparison of the disclosed techniques with state-of-the-art commercial supercontinuum systems (SuperK Extreme). In the disclosed system, the measured sensitivity is 105 dB and 6-dB sensitivity roll-off is 1.81 mm with 300 μW power on the sample at an A-line rate of 28 kHz. For comparison, a commercial supercontinuum system (SuperK Extreme) show 95 dB sensitivity and 1.25 mm 6-dB sensitivity roll-off with 4 mW of power on the sample at an A-line rate of 40 kHz.

Numerical Modeling of Supercontinuum Generation in Si₃N₄ Waveguide

FIG. 8 shows supercontinuum generation simulation in an example 5-cm-long Si₃N₄ waveguide. The top portion shows simulated spectra for 128 independent initiations. The bottom portion shows calculated first-order mutual coherence g₁₂.

FIG. 9 shows supercontinuum generation simulation with the example Si₃N₄ platform using a pump wavelength at 1.06 μm can emit a coherent spectrum spanning approximately 800 nm to 1400 nm. This simulated waveguide has a cross-section of 730 nm×700 nm and 6 mm long. The top portion shows simulated group velocity dispersion (GVD). The pump is centered at 1060 nm with 100 fs pulse duration and 75 pJ pulse energy. Middle: simulated 641 spectra for 128 independent initiations. The bottom portion shows calculated first-order mutual coherence.

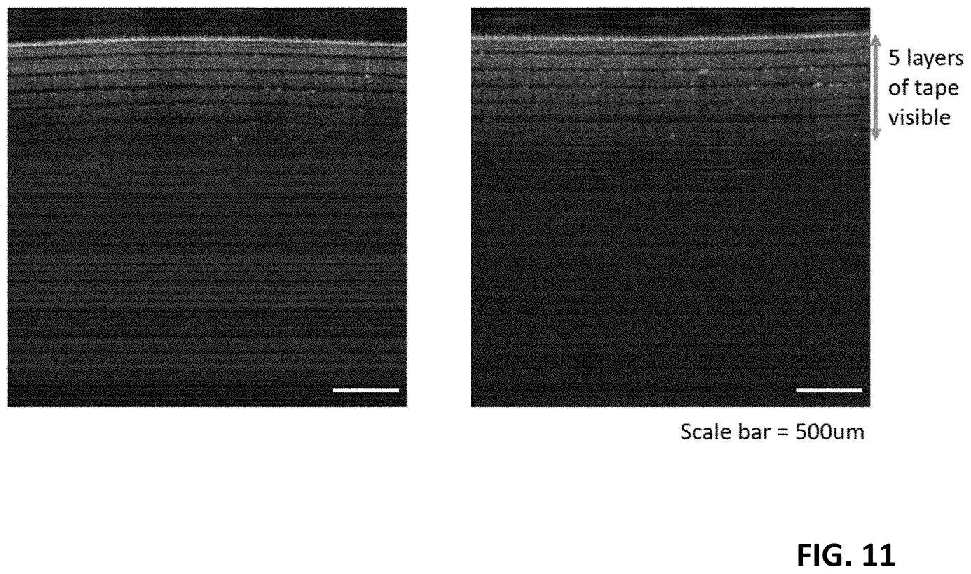

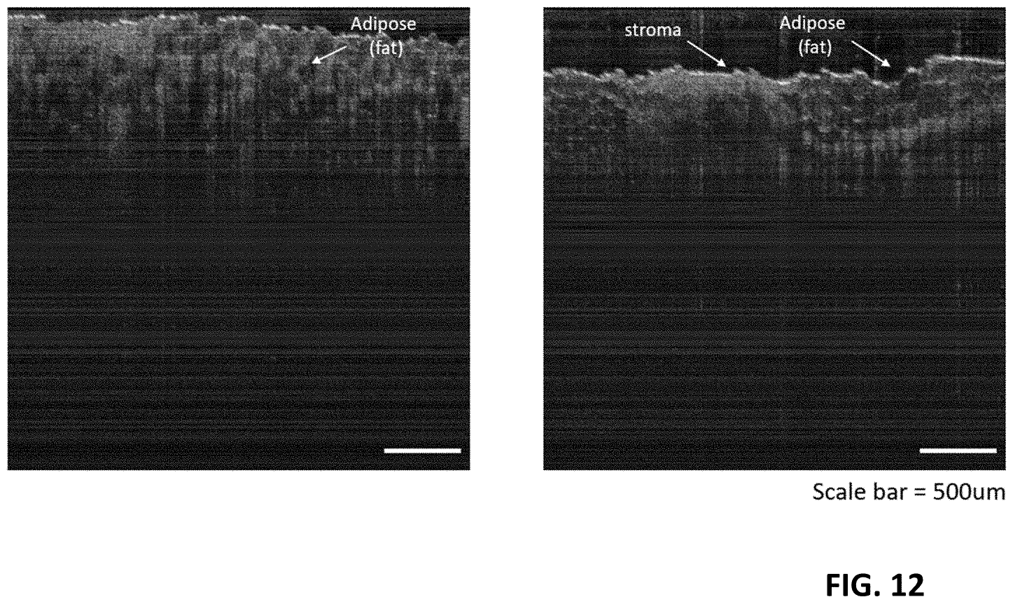

FIGS. 11-12 demonstrate that the disclosure may be performed at other wavelengths (e.g., centered at other wavelengths). FIG. 11 shows imaging of 10 layers of scotch tap of an example Si₃N₄ Chip-OCT system at 800 nm. FIG. 12 shows imaging of breast tissue by an example Si₃N₄ Chip-OCT system at 800 nm.

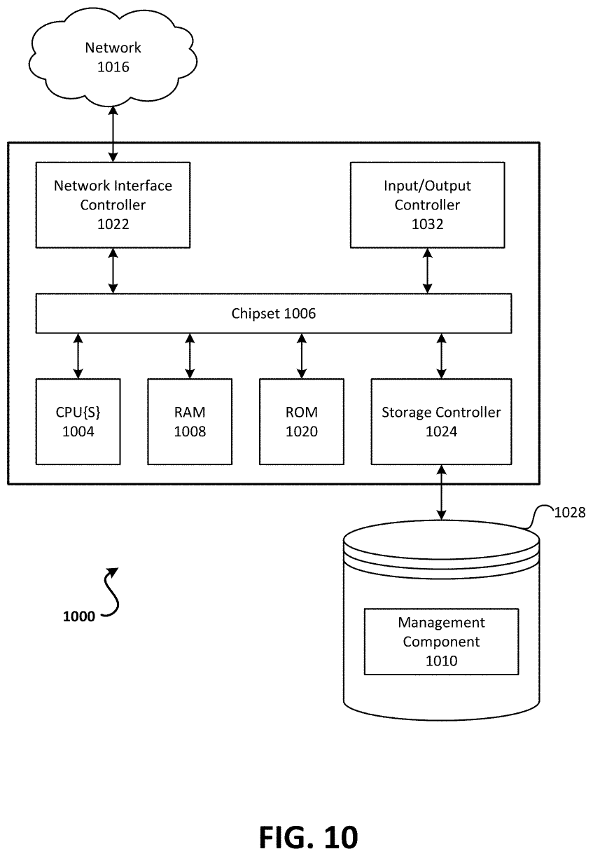

FIG. 10 depicts a computing device that may be used in various aspects, such as the devices and systems depicted herein. With regard to the example architecture of FIG. 3, the acquisition system, spectrometer, OCT scanner, pump laser, may each be implemented in an instance of a computing device 1000 of FIG. 10. The computer architecture shown in FIG. 10 shows a conventional server computer, workstation, desktop computer, laptop, tablet, network appliance, PDA, e-reader, digital cellular phone, or other computing node, and may be utilized to execute any aspects of the computers described herein, such as to implement the methods described herein.

The computing device 1000 may include a baseboard, or “motherboard,” which is a printed circuit board to which a multitude of components or devices may be connected by way of a system bus or other electrical communication paths. One or more central processing units (CPUs) 1004 may operate in conjunction with a chipset 1006. The CPU(s) 1004 may be standard programmable processors that perform arithmetic and logical operations necessary for the operation of the computing device 1000.

The CPU(s) 1004 may perform the necessary operations by transitioning from one discrete physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits including registers, adders-subtractors, arithmetic logic units, floating-point units, and the like.

The CPU(s) 1004 may be augmented with or replaced by other processing units, such as GPU(s) 1005. The GPU(s) 1005 may comprise processing units specialized for but not necessarily limited to highly parallel computations, such as graphics and other visualization-related processing.

A chipset 1006 may provide an interface between the CPU(s) 1004 and the remainder of the components and devices on the baseboard. The chipset 1006 may provide an interface to a random access memory (RAM) 1008 used as the main memory in the computing device 1000. The chipset 1006 may further provide an interface to a computer-readable storage medium, such as a read-only memory (ROM) 1020 or non-volatile RAM (NVRAM) (not shown), for storing basic routines that may help to start up the computing device 1000 and to transfer information between the various components and devices. ROM 1020 or NVRAM may also store other software components necessary for the operation of the computing device 1000 in accordance with the aspects described herein.

The computing device 1000 may operate in a networked environment using logical connections to remote computing nodes and computer systems through local area network (LAN) 1016. The chipset 1006 may include functionality for providing network connectivity through a network interface controller (NIC) 1022, such as a gigabit Ethernet adapter. A NIC 1022 may be capable of connecting the computing device 1000 to other computing nodes over a network 1016. It should be appreciated that multiple NICs 1022 may be present in the computing device 1000, connecting the computing device to other types of networks and remote computer systems.

The computing device 1000 may be connected to a mass storage device 1028 that provides non-volatile storage for the computer. The mass storage device 1028 may store system programs, application programs, other program modules, and data, which have been described in greater detail herein. The mass storage device 1028 may be connected to the computing device 1000 through a storage controller 1024 connected to the chipset 1006. The mass storage device 1028 may consist of one or more physical storage units. A storage controller 1024 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computing device 1000 may store data on a mass storage device 1028 by transforming the physical state of the physical storage units to reflect the information being stored. The specific transformation of a physical state may depend on various factors and on different implementations of this description. Examples of such factors may include, but are not limited to, the technology used to implement the physical storage units and whether the mass storage device 1028 is characterized as primary or secondary storage and the like.

For example, the computing device 1000 may store information to the mass storage device 1028 by issuing instructions through a storage controller 1024 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computing device 1000 may further read information from the mass storage device 1028 by detecting the physical states or characteristics of one or more particular locations within the physical storage units.

In addition to the mass storage device 1028 described above, the computing device 1000 may have access to other computer-readable storage media to store and retrieve information, such as program modules, data structures, or other data. It should be appreciated by those skilled in the art that computer-readable storage media may be any available media that provides for the storage of non-transitory data and that may be accessed by the computing device 1000.

By way of example and not limitation, computer-readable storage media may include volatile and non-volatile, transitory computer-readable storage media and non-transitory computer-readable storage media, and removable and non-removable media implemented in any method or technology. Computer-readable storage media includes, but is not limited to, RAM, ROM, erasable programmable ROM (“EPROM”), electrically erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage, other magnetic storage devices, or any other medium that may be used to store the desired information in a non-transitory fashion.

A mass storage device, such as the mass storage device 1028 depicted in FIG. 10, may store an operating system utilized to control the operation of the computing device 1000. The operating system may comprise a version of the LINUX operating system. The operating system may comprise a version of the WINDOWS SERVER operating system from the MICROSOFT Corporation. According to further aspects, the operating system may comprise a version of the UNIX operating system. Various mobile phone operating systems, such as IOS and ANDROID, may also be utilized. It should be appreciated that other operating systems may also be utilized. The mass storage device 1028 may store other system or application programs and data utilized by the computing device 1000.

The mass storage device 1028 or other computer-readable storage media may also be encoded with computer-executable instructions, which, when loaded into the computing device 1000, transforms the computing device from a general-purpose computing system into a special-purpose computer capable of implementing the aspects described herein. These computer-executable instructions transform the computing device 1000 by specifying how the CPU(s) 1004 transition between states, as described above. The computing device 1000 may have access to computer-readable storage media storing computer-executable instructions, which, when executed by the computing device 1000, may perform the methods described herein, such as generating an optical source for an OCT scanner, scanning using an OCT scanner, other imagine, analysis of scanned images, and/or the like.

A computing device, such as the computing device 1000 depicted in FIG. 10, may also include an input/output controller 1032 for receiving and processing input from a number of input devices, such as a keyboard, a mouse, a touchpad, a touch screen, an electronic stylus, or other type of input device. Similarly, an input/output controller 1032 may provide output to a display, such as a computer monitor, a flat-panel display, a digital projector, a printer, a plotter, or other type of output device. It will be appreciated that the computing device 1000 may not include all of the components shown in FIG. 10, may include other components that are not explicitly shown in FIG. 10, or may utilize an architecture completely different than that shown in FIG. 10.

As described herein, a computing device may be a physical computing device, such as the computing device 1000 of FIG. 10. A computing node may also include a virtual machine host process and one or more virtual machine instances. Computer-executable instructions may be executed by the physical hardware of a computing device indirectly through interpretation and/or execution of instructions stored and executed in the context of a virtual machine.

It is to be understood that the methods and systems are not limited to specific methods, specific components, or to particular implementations. It is also to be understood that the terminology used herein is for the purpose of describing particular embodiments only and is not intended to be limiting.

The disclosure includes at least the following aspects:

Aspect 1. A device comprising, consisting of, or consisting essentially of: a chip; and a waveguide disposed on the chip and comprising silicon nitride, wherein the waveguide is configured to generate, based on nonlinear effects applied to a pump signal from a pump laser, an optical signal having a broader spectrum than the pump signal, and wherein the waveguide has a width and a height such that the optical signal has near zero group-velocity-dispersion.

Aspect 2. The device of Aspect 1, wherein the pump signal is centered at a same frequency as the optical signal.

Aspect 3. The device of any one of Aspects 1-2, wherein one or more of the pump signal or the optical signal is centered at about 1300 nm.

Aspect 4. The device of any one of Aspects 1-3, wherein near zero group-velocity-dispersion comprises a group-velocity-dispersion in a range of about negative 50 ps/(nm km) to about positive 50 ps/(nm km).

Aspect 5. The device of any one of Aspects 1-4, wherein the optical signal has a wavelength imaging window centered at one or more of 800 nm, 1000 nm, 1300 nm, or 1700 nm.

Aspect 6. The device of any one of Aspects 1-5, wherein the optical signal does not require filtering for use by an optical coherence tomography (OCT) scanner.

Aspect 7. The device of any one of Aspects 1-6, wherein one of the width or height is 840 nm and the other of the width or height is 730 nm.

Aspect 8. The device of any one of Aspects 1-7, wherein one of the height is in a range of about 600 nm to about 900 nm and the other of the width is in a range of about 700 nm to about 3000 nm.

Aspect 9. The device of any one of Aspects 1-8, wherein the chip has an area equal to or less than one or more of about 1 cm², about 10 mm², about 1 mm², or about 0.25 mm².

Aspect 10. The device of any one of Aspects 1-9, wherein the optical signal comprises a broad spectral bandwidth through supercontinuum generation. A supercontinuum may be formed when a collection of nonlinear processes act together upon a pump beam in order to cause severe spectral broadening of the original pump beam.

Aspect 11. The device of any one of Aspects 1-10, wherein the waveguide has a length in a range of one or more of about 2-3 cm, about 5 cm, or about 2 cm to about 100 cm.

Aspect 12. A system comprising, consisting of, or consisting essentially of: a pump laser configured to generate a pump signal; an optical coherence tomography (OCT) scanner; and the device of any one of Aspects 1-11, wherein the device is configured to generate an optical signal based on the pump signal and provide the optical signal to the OCT scanner.

Aspect 13. The system of Aspect 12, further comprising an acquisition system configured to one or more of control the OCT scanner, process an image from the scanner generated based on the optical signal, or control the pump laser.

Aspect 14. A method comprising, consisting of, or consisting essentially of: generating, by a pump laser, a pump signal; providing the pump signal to a waveguide disposed on a chip, wherein the waveguide comprises silicon nitride; and generating, based on nonlinear effects caused by the waveguide to the pump signal, an optical signal having a broader spectrum than the pump signal, and wherein the waveguide has a width and a height such that the optical signal has near zero group-velocity-dispersion.

Aspect 15. The method of Aspect 14, further comprising supplying the optical signal to an optical coherence tomography (OCT) scanner.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Components are described that may be used to perform the described methods and systems. When combinations, subsets, interactions, groups, etc., of these components are described, it is understood that while specific references to each of the various individual and collective combinations and permutations of these may not be explicitly described, each is specifically contemplated and described herein, for all methods and systems. This applies to all aspects of this application including, but not limited to, operations in described methods. Thus, if there are a variety of additional operations that may be performed it is understood that each of these additional operations may be performed with any specific embodiment or combination of embodiments of the described methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded on a general-purpose computer, special-purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto may be performed in other sequences that are appropriate. For example, described blocks or states may be performed in an order other than that specifically described, or multiple blocks or states may be combined in a single block or state. The example blocks or states may be performed in serial, in parallel, or in some other manner. Blocks or states may be added to or removed from the described example embodiments. The example systems and components described herein may be configured differently than described. For example, elements may be added to, removed from, or rearranged compared to the described example embodiments.

It will also be appreciated that various items are illustrated as being stored in memory or on storage while being used, and that these items or portions thereof may be transferred between memory and other storage devices for purposes of memory management and data integrity. Alternatively, in other embodiments, some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

While the methods and systems have been described in connection with preferred embodiments and specific examples, it is not intended that the scope be limited to the particular embodiments set forth, as the embodiments herein are intended in all respects to be illustrative rather than restrictive.

It will be apparent to those skilled in the art that various modifications and variations may be made without departing from the scope or spirit of the present disclosure. Other embodiments will be apparent to those skilled in the art from consideration of the specification and practices described herein. It is intended that the specification and example figures be considered as exemplary only, with a true scope and spirit being indicated by the following claims.

What is claimed:

1. A device comprising:
a chip; and
a waveguide disposed on the chip and comprising silicon nitride, wherein the waveguide is configured to generate, based on nonlinear effects applied to a pump signal from a pump laser, an optical signal having a broader spectrum than the pump signal, and wherein the waveguide has a width and a height such that the optical signal has near zero group-velocity-dispersion.

2. The device of claim 1, wherein the pump signal is centered at a same frequency as the optical signal.

3. The device of claim 1, wherein one or more of the pump signal or the optical signal is centered at about 1300 nm.

4. The device of claim 1, wherein near zero group-velocity-dispersion comprises a group-velocity-dispersion in a range of about negative 50 ps/(nm km) to about positive 50 ps/(nm km).

5. The device of claim 1, wherein the optical signal has a wavelength imaging window centered at one or more of 800 nm, 1000 nm, 1300 nm, or 1700 nm.

6. The device of claim 1, wherein the optical signal does not require filtering for use by an optical coherence tomography (OCT) scanner.

7. The device of claim 1, wherein one of the width or height is 840 nm and the other of the width or height is 730 nm.

8. The device of claim 1, wherein one of the height is in a range of about 600 nm to about 900 nm and the other of the width is in a range of about 700 nm to about 3000 nm.

9. The device of claim 1, wherein the chip has an area equal to or less than one or more of about 1 cm², about 10 mm², about 1 mm², or about 0.25 mm².

10. The device of claim 1, wherein the optical signal comprises a broad spectral bandwidth through supercontinuum generation.

11. The device of claim 1, wherein the waveguide has a length in a range of one or more of about 2-3 cm, about 5 cm, or about 2 cm to about 100 cm.

12. A system comprising:
a pump laser configured to generate a pump signal;
an optical coherence tomography (OCT) scanner; and
a device configured to generate an optical signal based on the pump signal and provide the optical signal to the OCT scanner, wherein the device comprises:
a chip; and
a waveguide disposed on the chip and comprising silicon nitride, wherein the waveguide is configured to generate, based on nonlinear effects applied to the pump signal, the optical signal, wherein the optical signal has a broader spectrum than the pump signal, and wherein the waveguide has a width and a height such that the optical signal has near zero group-velocity-dispersion.

13. The system of claim 12, further comprising an acquisition system configured to one or more of control the OCT scanner, process an image from the scanner generated based on the optical signal, or control the pump laser.

14. A method comprising:
generating, by a pump laser, a pump signal;
providing the pump signal to a waveguide disposed on a chip, wherein the waveguide comprises silicon nitride; and
generating, based on nonlinear effects caused by the waveguide to the pump signal, an optical signal having a broader spectrum than the pump signal, and wherein the waveguide has a width and a height such that the optical signal has near zero group-velocity-dispersion.

15. The method of claim 14, further comprising supplying the optical signal to an optical coherence tomography (OCT) scanner.
